(19) United States
(12) Patent Application Publication
(10) Pub. No.: US 2012/0226183 A1
(43) Pub. Date: Sep. 6, 2012

(54) EVACUATED AIR CHAMBER

(75) Inventors: NORBERT JOHN THOMAS CHRISTMAN, Wauwatosa, WI (US); Steven D. Hamilton, Greenfield, WI (US); Katherine M. Ross, West Allis, WI (US)

(73) Assignee: QUINTRON INSTRUMENT COMPANY, Milwaukee, WI (US)

(21) Appl. No.: 13/221,446

(22) Filed: Aug. 30, 2011

Related U.S. Application Data
(60) Provisional application No. 61/402,464, filed on Aug. 30, 2010.

(51) Int. Cl. A61B 5/097

(52) U.S. Cl. 600/543

(57) ABSTRACT

An improved evacuated air chamber having a plurality of generally flat exterior walls, a closed end, and an open end wherein the open end is provided with a resealable stopper and septum; an impervious disc may be provided between the septum and the tube interior.

### RELATED APPLICATIONS

This application claims the benefit of U.S. Provisional Application Ser. No. 61/402,464 filed 30 Aug. 2010.

### BACKGROUND OF THE INVENTION

The present invention relates to the field of evacuated air chambers, in particular those used to contain samples of air from the lungs, such as those used to contain alveolar air from the alveoli of the lungs of a person.

Air from the lungs of a person can be used for many different types of testing that would otherwise require the person to undergo an invasive procedure. For example, alveolar air can be analyzed for the noninvasive diagnosis of a wide variety of conditions including stomach infection related to a high incidence of ulcers, enzymatic deficiencies, and metabolic conditions and/or abnormalities. Crucial to any such testing is the ability to obtain an accurate and untainted sample containing a sufficient volume of air representative of true alveolar air, necessary for a specific testing.

Typical air sampling devices or breath collection apparatuses with which the present device may be used include a breath intake structure, an expandable bag connected to the breath intake structure by a connecting means, an air sample capture assembly selectively detachable from an expandable bag, and an evacuated air chamber, such as a tube, having a rubber or other resilient material membrane stopper capable of being punctured when coupled with said air capture assembly and resealed when uncoupled. The present invention includes specific improvements to the evacuated air chamber for use with such a device. Included are improvements in the chamber internal surface configuration, external surface configuration and stopper configuration.

While the present invention is particularly suited for containing air samples obtained using a breath collection apparatus, it is to be understood that the improved evacuated air chamber may have other uses in the medical sampling arena.

Known evacuated air chambers are typically of a tubular form, such as a test tube, however there are several disadvantages to this usual design, as will be discussed. Since samples captured and contained in tubular evacuated air chambers typically undergo subsequent testing, such as gas or liquid analysis, it is critical that the samples contained in the evacuated air chambers are kept in pristine condition to thereby enable accurate testing with no contaminates influencing test results. However, contamination of traditional tube type chambers has been known to occur. For example, prior to use, present tube-form evacuated air chambers are typically shipped and stored in vacuum sealed packaging. The rounded external surface contour of the typical tube shape permits interstitial air pockets to form around the tube body exterior while encased by packaging. These pockets may permit cross contamination of the evacuated interior with the atmospheric air surrounding the tube in the pocket areas. Any contamination of the evacuated interior is undesirable since sample accuracy and consistency may be affected. Further, traditional tubes may roll and cause breakage. In addition, known evacuated tubes may be difficult to set up for fraction collection.

Another disadvantage of the typical evacuated tube design is related to the stopper structure through which a sample is drawn. A typical evacuated tube includes a cap or stopper having a septum or other like self sealing structure through which samples may be drawn. In some situations, the septum has been known to absorb an amount of CO₂. This is undesirable since CO₂ absorption by the septum may affect accurate content sampling. The present configuration resolves these difficulties while providing further advantages to the present tube design.

The present device contemplates improvements to the tube internal and tuber exterior tube configurations to minimize the formation of interstitial air pockets while packaged. The present device further contemplates improvements in stopper configuration. The present invention aims to decrease sample contamination to thereby increase sample purity.

### SUMMARY OF THE INVENTION

The present invention is directed to an improved evacuated air chamber, or tube, used to contain samples, such as alveolar air samples, and to be used in conjunction with collection apparatuses, such as a breath collection apparatus, for example.

Specifically, the present invention is directed to an improved evacuated air chamber preferably having a plurality of generally flat exterior walls, a hollow interior, a closed end, and an open end wherein the open end is preferably provided with a resealable stopper and pierceable septum. The air chamber exterior has generally square cross section, while the interior may have a polygonal cross section. Further, the stopper member may include an impervious disc located between the septum and the tube interior.

Collection devices for use in conjunction with the present invention may include a typical breath collection device, for example. An illustrative collection device may include an initial capture device, such as an expandable bag, which is filled with a sample of alveolar air. The initial capture device can be repeatedly sampled by simply placing an evacuated air chamber onto an air capture assembly.

As previously mentioned, the generally flat exterior walls of the present design allow a more effective vacuum seal while packaged and during storage since interstitial air pockets formed between the packaging and the tube exterior wall surfaces are minimized. As discussed, the minimization of interstitial space around the tubes while packaged results in a reduced likelihood of chamber contamination due to reduced exterior wall contact with surrounding air. Further, the square design permits a larger sample volume and enables a more facile set up for fraction collection. The flat exterior walls also provide an easy surface on which to write or place a label. Low profile grips may be provided on the exterior walls for ease of use, if desired.

The present invention further contemplates an improved cap for placement over the open end, in which an impermeable disc may be placed between the septum and the sample, thereby reducing CO₂ absorption by the septum and providing more accurate sample contents.

The typical sample contained by the present device is captured in a vacuum state by the improved evacuated air chamber, and is then tested as desired. It is to be understood that the present evacuated air chamber may contain different agents, or may be of different volumes, to thus allow different chemical tests, tags, and analyses to be conducted on the same sample thereby enhancing the consistency and accuracy of the test information so gathered.

### BRIEF DESCRIPTION OF THE DRAWINGS

FIG. 1 is a perspective view of an illustrative breath collection apparatus for use with the present invention.

FIG. 2 is a perspective view of an evacuated air chamber according to the present invention.

FIG. 3A is a cross sectional view of the chamber shown in FIG. 1 and taken along lines 3A-3A thereof.

FIG. 3B is a cross sectional view of the chamber shown in FIG. 2, and taken along lines 3B-3B thereof but showing an alternative interior wall configuration.

FIG. 4 is a longitudinal sectional view of the chamber illustrated in FIG. 2 and taken along lines 4-4 thereof.

FIG. 4A is a fragmentary, enlarged view of a portion of the device shown in FIG. 4.

FIG. 5 is a perspective view of the device illustrated in FIG. 2, but showing a protective cover in place.

FIG. 6A is a perspective view of a prior art evacuated tube encased in packaging and showing the resultant interstitial air pockets.

FIG. 6B is a view similar to that of FIG. 6A, but showing an embodiment of the present invention in packaging wherein interstitial space is greatly reduced or eliminated.

FIG. 7 is a perspective view of multiple evacuated tubes according to the present invention as arranged for fraction collection.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

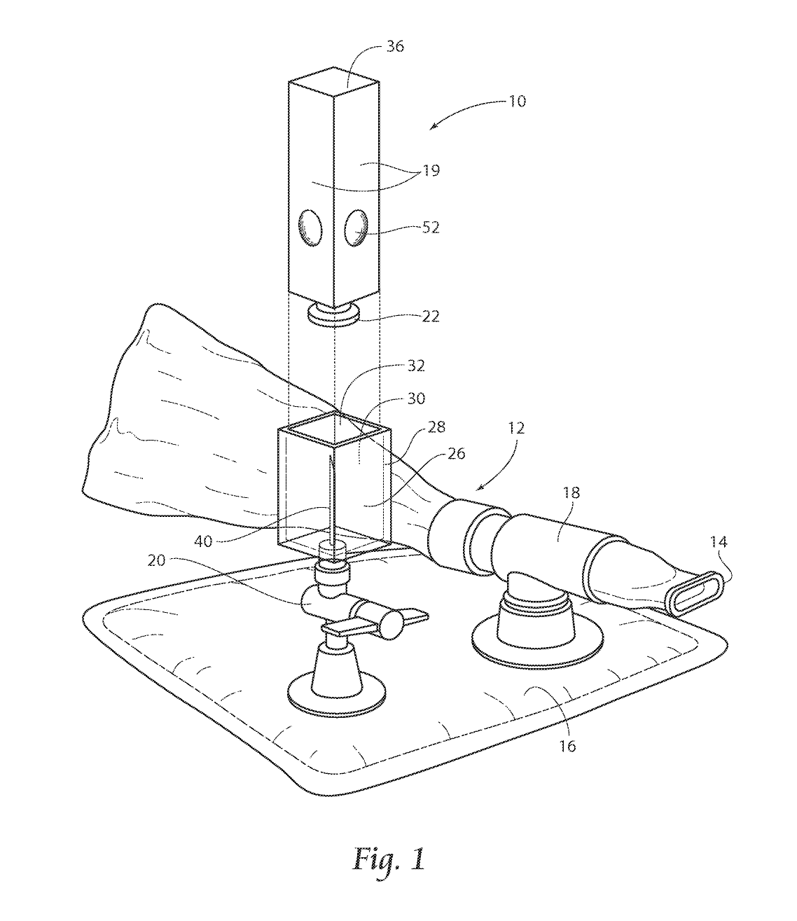

As seen in the Figures, the present invention is directed to an improved evacuated air chamber 10 and method of use. As previously mentioned, the present air chamber 10 may be used in conjunction with sample collection apparatuses, such as the breath collection apparatus 12 shown in FIG. 1, although it is to be understood that other uses for the chamber 10 may be envisioned.

With specific reference to FIG. 1, the evacuated air chamber 10 may be seen in conjunction with an illustrative breath collection apparatus 12. As shown, the illustrative breath collection apparatus 12 may typically include a breath intake structure 14, an expandable bag 16 connected to the breath intake structure 14 by a connecting means 18, and an air sample capture assembly 20 selectively detachable from an expandable bag 16. With further reference to FIG. 1, a typical air capture assembly 20 for use with the air chamber 10 may be seen to also include a needle assembly holder 26 comprising a receptacle 28 having a hollow interior 30, and a large open end 32 designed to receive the evacuated chamber 10 into the hollow interior 30. Although suitable results have been achieved by using Beckton Dickinson products marketed under the “VACUTAINER Brand” for the needle assembly holder 26, it is to be noted that the needle assembly holder 26 illustrated in these views is modified to fit the novel evacuated chamber 10.

The evacuated air chamber 10 of the present invention may be seen in the Figures as having a plurality of relatively flat exterior walls 19, a hollow interior 24, a closed end 36, and an open end 34 wherein the open end 34 is preferably provided with a resealable stopper member 22. The stopper member 22 may be formed of rubber or other resilient material that is capable of being punctured when coupled with the air capture assembly 20 and resealed after removal. The stopper member 22 preferably includes a pierceable septum 38 and impervious disc 42 located between the septum 38 and the tube interior 24, as will be discussed below.

Use of the present evacuated air chamber 10 in combination with a collection apparatus 12 may be further seen in FIG. 1. As is illustrated, the evacuated air chamber 10 is pushed down into the receptacle 28 of a needle assembly holder 26 so that the stopper member 22, septum 38, and disc 42 are pierced by the needle 40. As the stopper member 22 and septum 38 are pierced a predetermined vacuum contained within the evacuated chamber interior 24 supplies the predetermined suction necessary to draw a sufficient sample of captured air from the expandable bag 16. The evacuated chamber 10 is then withdrawn from the receptacle 28 and the resilient rubber stopper member 22 and septum 38 will reseal to protect and contain the sample drawn.

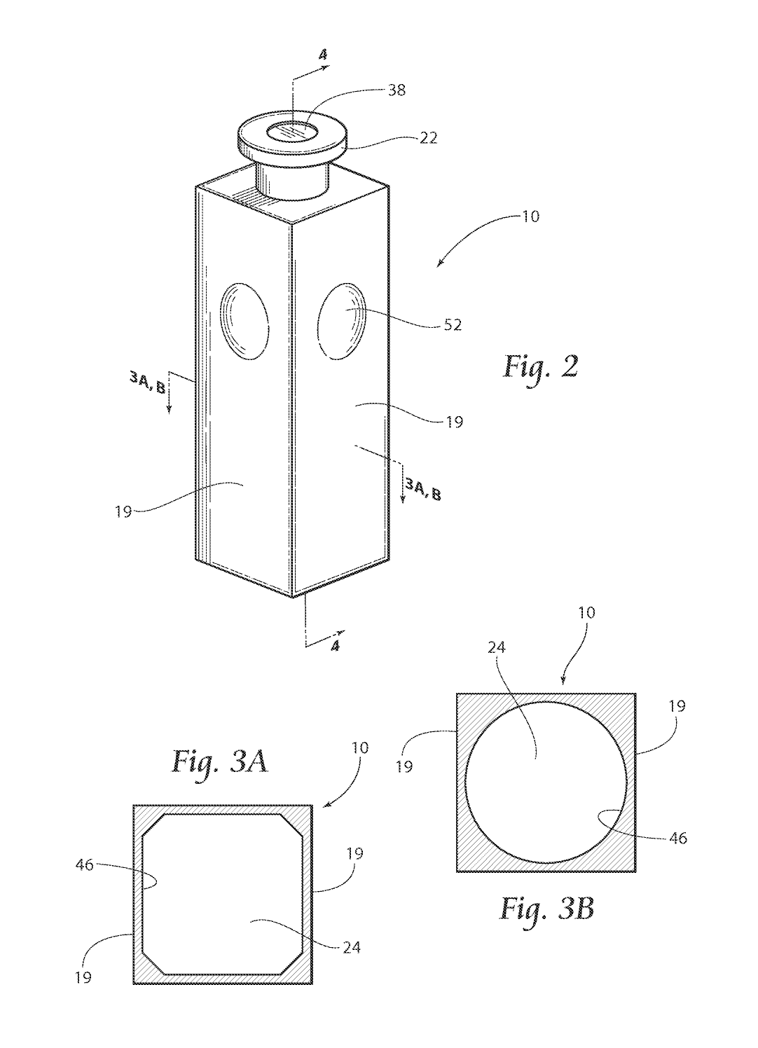

Still referring to FIG. 1, but also to FIGS. 2-6, the evacuated air chamber 10 of the present invention is seen as having an open end 34 and a closed end 36, with the open end 34 including a stopper member 22 made of rubber or some other sufficiently resilient material. A pierceable septum 38 is also preferably provided to maintain the sample prior to extraction for testing. Of particular note are the relatively flat exterior walls 19, which impart many of the discussed advantages of the present device 10. The relatively flat exterior walls 19 may be optionally provided with depressions or low profile grips 52 for ease of handling in use, if desired. The depressions 52 may be placed anywhere on the exterior walls 19, but are optimally placed above the chamber 10 center of gravity for facile balance. In a preferred embodiment, the evacuated air chamber 10 is comprised of glass or metal. Unlike air chambers made of other materials, such as plastic, tubes made of glass or metal are impermeable to hydrogen (H₂). This is an important consideration since permeability to molecular hydrogen may affect certain types of chemical tests or analysis.

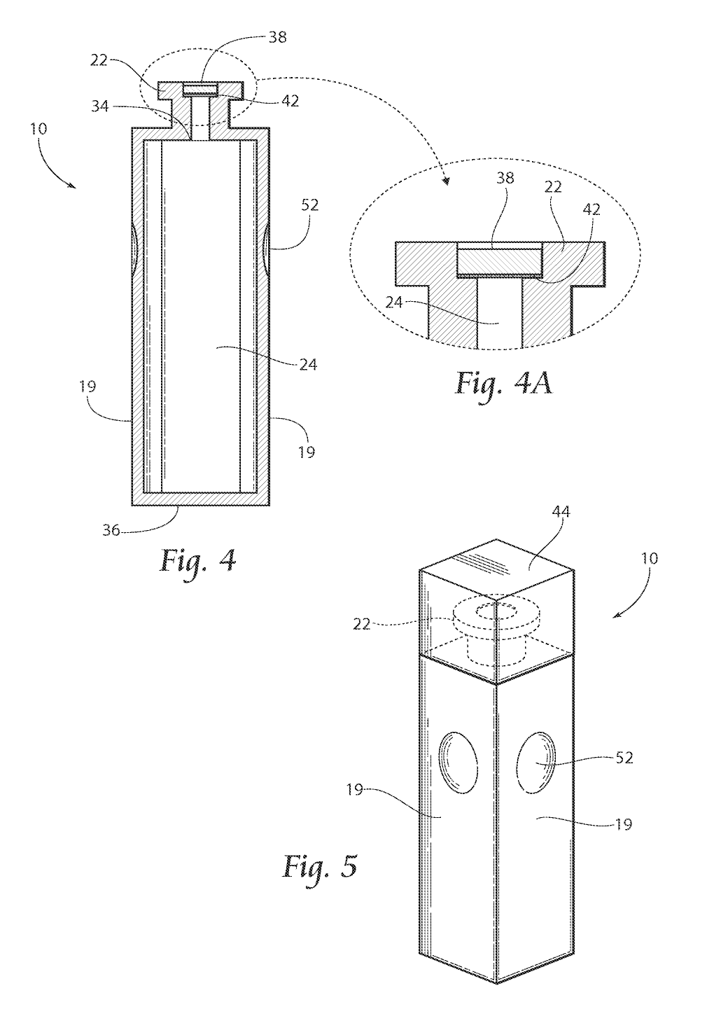

As illustrated particularly in the views of FIGS. 4 and 4A, a disc 42 of TEFLON®, foil, or other suitable material may be placed between the septum 38 and the sample. The disc 42 provides a barrier between the sample contained in the chamber 10 and the septum 38 to thereby prevent CO₂ absorption by the septum. In known arrangements, the absorption of CO₂ by the septum 38 may increase the likelihood of sample contamination by CO₂. In contrast, the present device, with the included barrier disc 42 further preserves the integrity of the sample within the chamber 10 by reducing potential CO₂ contamination.

Shown in FIG. 5 is an additional cover 44 or seal that may be placed optionally over the rubber stopper member 22 to ensure that the evacuated chamber 10 is properly sealed. The cover 44 may further reduce the likelihood of chamber 10 contamination during storage and shipping. Further, it should be noted that the evacuated air chamber 10 could be preloaded with desiccant or a chemical indicator (not shown) for a variety of tests in addition to those tests specifically disclosed herein.

With specific reference now to FIGS. 3A and 3B, it may be seen that an internal surface 46 of the chamber 10 may have various cross section configurations. FIG. 3A, for example illustrates an internal surface having a polygonal cross section. The view of FIG. 3B depicts a generally circular cross section. It is to be understood that the internal surface 46 may have various configurations in which a sample volume may be accurately captured and tested.

FIGS. 4 and 4A illustrate an arrangement of stopper member 22, septum 38 and disc 42. As previously noted, the stopper member 22 and septum 38 are preferably fabricated of a material known to reseal after puncture to thereby protect and contain the sample drawn. As shown, a disc 42 of TEFLON®, foil, or other suitable material may be placed between the septum 39 and the sample, to thereby prevent CO₂ absorption by the septum and to further ensure sample purity.

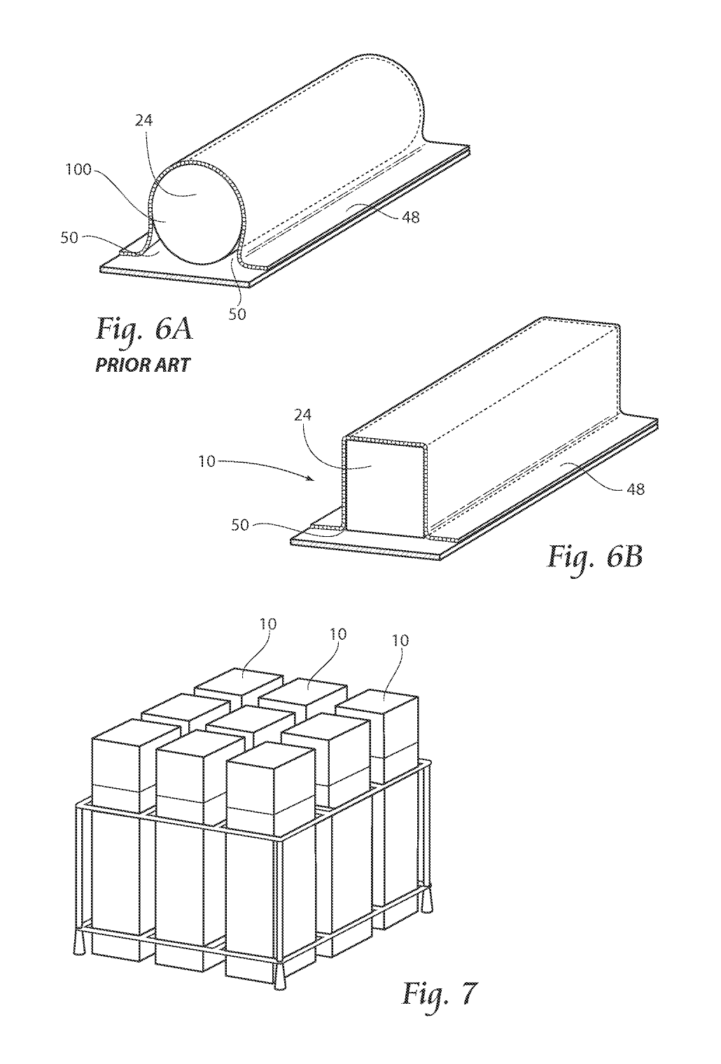

With reference now to FIGS. 6A and 6B, a further advantage of the present evacuated tube 10 is illustrated. The prior art tube 100 depicted in FIG. 6A is seen in vacuum-sealed packaging 48 typical for storage and shipping. As seen, the packaging 48 cooperates with the curved outer tube 100 surface to form interstitial areas 50. The interstitial areas 50 created may contain atmospheric air and permit cross contamination of the evacuated interior 30 with atmospheric air. As previously mentioned, any contamination of the evacuated interior 30 is undesirable since sample accuracy and consistency may be affected. Illustrated in FIG. 6B is the present evacuated chamber 10 having relatively flat exterior walls 19. The unique exterior wall 19 configuration allows packaging 48 to be vacuum-sealed securely adjacent the tube 10 walls 19. This arrangement greatly minimizes or eliminates the surrounding interstitial area 50. Reduction of interstitial area 50 reduces the chamber 10 exterior wall 19 surface area in contact with atmospheric air therefore reducing potential contamination of the interior 30.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention, which is defined by the claims.

We claim:

1. An evacuated air chamber having a plurality of generally flat exterior walls, a hollow elongated evacuated interior, a first, closed end, and a second, open end, wherein said second end is provided with a resealable stopper member normally enclosing said second end.

2. The chamber of claim 1, wherein said stopper member further includes a pierceable septum member and an impervious disc member, said disc being positioned between the septum member and said interior.

3. The chamber of claim 1 wherein said evacuated interior includes an interior wall surface, said interior wall surface having a polygonal cross section.

4. The chamber of claim 1 wherein said evacuated interior includes an interior wall surface, said interior wall surface having an arcuate cross section.

5. The chamber of claim 1 wherein at least one of said plurality of exterior walls includes a depression.
